(19) United States
(12) Patent Application Publication
(10) Pub. No.: US 2002/0127597 A1
(43) Pub. Date: Sep. 12, 2002

(54) Method and apparatus for exploring an experimental space

(75) Inventors: Yu-To Chen, Niskayuna, NY (US); Robert Marcel Mattheyses, Schenectady, NY (US); James Norman Cawse, Pittsfield, MA (US); Christopher Leon Stanard, Niskayuna, NY (US); Carl Harold Hansen, Latham, NY (US)

Correspondence Address: GENERAL ELECTRIC COMPANY, CRD PATENT DOCKET ROOM 4A59, P O BOX 8, BUILDING K 1 SALAMONE, SCHENECTADY, NY, 12301, US

(73) Assignee: General Electric Company

(21) Appl. No.: 09/757,246

(22) Filed: Jan. 10, 2001

(51) Int. Cl. G01N 33/53; G06G 7/48; G06G 7/58; G06F 19/00

(52) U.S. Cl. 435/7.1; 702/19; 703/11

(57) ABSTRACT

A hybrid learning system is provided for searching an experimental space. A data mart is configured to acquire, store, and manipulate a set or meta-set of data including at least historical experimental data, descriptor data, and concurrent experimental data. A search engine is designed to use unsupervised learning techniques to select a set of evaluation points representing a corresponding set of experiments to be run, based on data from the data mart. A point evaluation mechanism provided with supervised learning modules which perform predictive processing based on the evaluation points selected by the search engine, and a scoring module performs a rating operation on outputs of the learning modules to rate the outputs of the learning modules from best to worst. The data mart search engine and point evaluation mechanism allow for a repetitive processing to refine an output of potential solutions without the requirement of continually running actual physical experiments.

### FIELD OF THE INVENTION

The present application relates to searching an experimental space of potential experiments related to material development in an effective and parsimonious manner.

### BACKGROUND OF THE INVENTION

In performing research for new innovations, researchers commonly look for useful synergies and/or interactions between multiple elements arranged in a variety of combinations. One area where these types of experiments are undertaken is the area of Combinatorial Chemistry. The discussion in the present application will focus on this area. It is however to be appreciated that the concepts of the present application may be extended to other areas where large numbers of various combinations of items are being tested.

The expansion of Combinatorial Chemistry has led to ever larger sizes of experimental spaces. As an example, consideration is given to the problem of finding a binary catalyst system where the binary catalysts are all chosen from a set of 22 individual candidates, each to be used at a single fixed concentration. For this problem, there are Choose (22,2) or 231 experiments. If this problem is simply altered to look at three different concentrations for each catalyst in a combination, the number of experiments is increased to Choose (22,2)×3², or 2079 experiments. Another problem is considered where a system is investigated consisting of three metals in combination with two anions. The metals are chosen from a set of 20 candidates, and the anions are chosen from a set of 20 candidates. Each component, metal, and anion can appear in any one of three concentrations, low, medium, or high. These parameters would lead to an experimental space of a size Choose (20,3)×3³×Choose (20,2)×3² or 52,633,800 possible experiments. The foregoing illustrates that investigations in Combinatorial Chemistry can span a very wide range, where no single approach can address each individual problem.

In addition to size, another factor influencing the effort required to search an experimental space is the time necessary to evaluate a point in the space, where a point corresponds to a potential solution. Although several points can be examined at once, each point requires time for setup, run, measurement and recording of experimental results. The present upper limit of concurrent experimentations for Combinational Chemistry is 110. It is further noted that the experiment cycle can commonly require on the order of one to several days. Using a cycle time of one day, the binary catalyst problem of the previous paragraph can be completed in three days. This is quite acceptable. However, for the more complex system, with three metals and two anions, assuming 250 work days in a year, slightly more than 1913 years would be required to cover the entire experimental space.

Thus, an area where improvement in experimentation processes is desirable is where the size of a problem grows rapidly and becomes too large for an exhaustive search to be applied. Also of interest is how can an experimental space be managed to allow effective and efficient development experiments to be performed in a parsimonious manner.

### BRIEF SUMMARY OF THE INVENTION

A hybrid learning system is provided for searching an experimental space. A data mart is configured to acquire, store, and manipulate a set or meta-set of data including at least historical experimental data, descriptor data, and concurrent experimental data. A search engine is designed to use selection techniques to select a set of evaluation points representing a corresponding set of experiments to be run, using data from the data mart. A point evaluation mechanism provided with supervised learning modules which perform predictive processing based on the evaluation points selected by the search engine, and a scoring module performs a rating operation on outputs of the learning modules to rate the outputs of the learning modules from best to worst. The data mart, search engine, and point evaluation mechanism allow for repetitive processing to refine an output of potential solutions without the requirement of continually running actual physical experiments.

### BRIEF DESCRIPTION OF THE DRAWINGS

FIG. 1 sets forth a schematic diagram of the hybrid learning system according to the present invention;

FIG. 2 is a table of sample data for a triple catalyst run;

FIG. 3 is a table of sample data for chemical descriptors;

FIG. 4 is a schematic representing the flow of the operation for controlling a search in accordance with the present invention;

FIG. 5 is a graphical representation of an experimental space which is being searched in the present invention;

FIG. 6 is a flow diagram representing unsupervised learning process of the present invention;

FIG. 7 depicts an experimental space divided in accordance with the clustering concept;

FIG. 8 depicts a repartitioned experimental space in accordance with fuzzy clustering;

FIG. 9 depicts a flow diagram of a genetic algorithm search process;

FIG. 10 illustrates the partitioning of an experimental space in accordance with the operation of a genetic algorithm;

FIG. 11 illustrates a table representing elements of the fitness function in accordance with the present invention; and

FIG. 12 sets forth a flow diagram for a supervised learning process.

### DETAILED DESCRIPTION OF INVENTION

The manner in which a search of an experimental space is to be undertaken is influenced by the size of the space. For smaller problems, it may be effective to investigate all points within a space, while for others certain rules of searching the space need to be provided. As a rough guide, the present embodiment classifies an experimental space by the size of the space, using one month, 20 working days, as an upper limit and an experimental cycle time of one day. Using this criteria, a first class of problems are identified as a small experimental space when they have fewer than 2K points which may be investigated. The next limit is determined by the ability to superimpose experiments without creating artifacts. As an example of super-positioning, the search of a ternary catalyst system is considered. For this type of search it is presumed the experiment will have five catalysts in a single experimental vial. This experiment could therefore be considered as looking at Choose (5,3), or ten three-element systems simultaneously. Critical to the success of this method is the requirement that the ten simultaneous experiments in the single vial do not interfere with each other to obscure the performance of the individual ternary systems. Consider a practical upper limit of the number of experiments that can be performed simultaneously in a single vial to be 50. Also, consider practical upper limit on the number of vials that can be processed in a reasonable amount of time to be 2000. In this case, an upper limit to the size of a space that can be considered using an exhaustive search with superposition of experiments will consist of 100,000 points of investigation. This will be considered as an upper limit for medium sized problems. Therefore, all problems of more than 100 k points are classified as large experimental spaces. However, it is to be appreciated that some spaces of fewer than 100 k points, where packing is prohibited, may also be treated as large.

Table 1 sets forth the combinatorial search problem classifications previously presented. It is to be appreciated that problems may be categorized as small, medium, or large for different numbers of points to be investigated. Further, the spaces may be categorized by designations other than small, medium or large spaces.

TABLE 1

| Lower | Upper | Class | Approach |
|---|---|---|---|
| — | 2K | Small | Exhaustive consideration |
| | | | of all experiments |
| 2K | 100K | Medium | Superposition of experiments |
| 100K | — | Large | Intelligent search management |

For large problems (i.e. 100k or more points to be tested), search management becomes a critical factor since only a small fraction of the experimental space can realistically be considered. Thus, the present invention is particularly interested in managing the experimental space for experiments classified as large.

To accomplish the foregoing, a hybrid of search techniques are brought together in concert to manage the search of an experimental space, such as a Combinatorial Chemistry experimental space (CC-space). This hybrid search apparatus and method builds on the concepts disclosed in U.S. patent Ser. No. 09/595,005 to Cawse et al., filed Jun. 16, 2000 entitled High Throughput Screening Method And System, hereby incorporated by reference. The Cawse et al. application connects the logical process of generating a next search set, by use of a search process such as a genetic algorithm, with a physical experiment being undertaken. A concept discussed in U.S. Ser. No. 09/595,005 is that in a basic genetic algorithm, when a space is defined by a mathematical function, “good points” are generated using standard genetic algorithm techniques and attempts are then made to evaluate mathematical functions at those points. However, in U.S. Ser. No. 09/595,005, what is being considered is not mathematical functions, but rather a physical system which is to be used as the evaluation of the net worth of the good points provided. Therefore, U.S. Ser. No. 09/595,005 takes the basic idea of generating evaluation points using genetic algorithm techniques and couples that concept with a physical experiment.

However, the present invention acknowledges that experiments may be expensive to perform, both in terms of time and economics. It is therefore considered desirable to run the genetic algorithm (or other selection processes, such as clustering) using data obtained not only from physical experiments but also data gained from a synthetic model, in order to obtain an improved set of evaluation points investigated. The present invention builds on U.S. Ser. No. 09/595,005 by implementing space management techniques to construct, from the data available, a best model possible of the CC-space under investigation. The selection processes may be run for several cycles before producing a set of proposed points at which to perform experiments to obtain more data to place back into the system for further refinement.

Thus, each time a set of experiments is performed, additional data is added to the system and a further refined model is generated. The selection processes are then again run against the new improved model. This interactive repetitive technique is undertaken a predetermined number of cycles by a user.

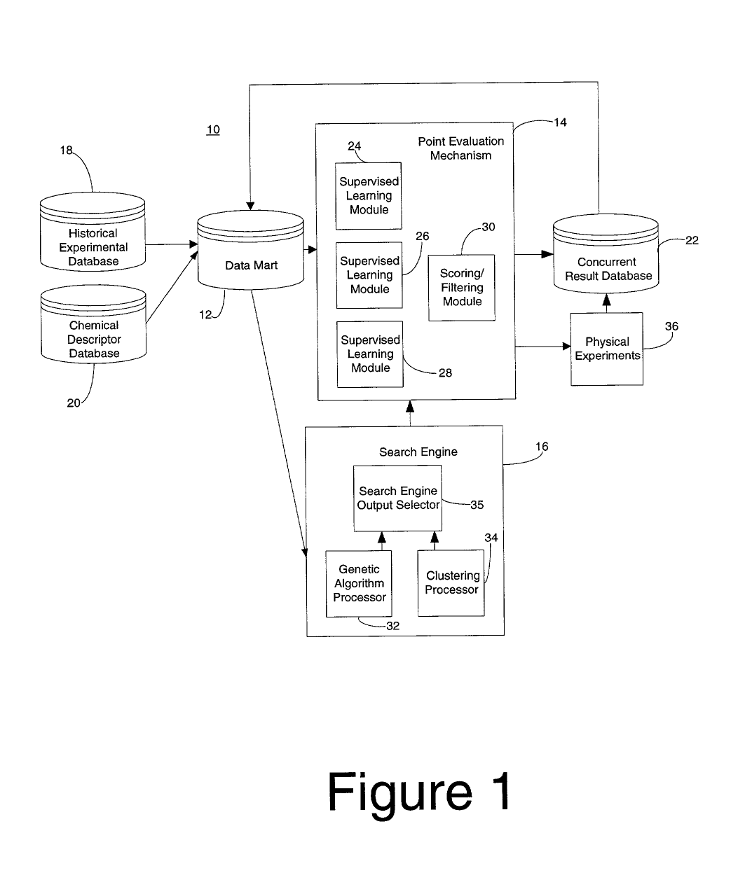

FIG. 1 diagrams a hybrid learning system 10 according to the concepts of the present invention. Hybrid learning system 10 includes at least a data mart 12, a point evaluation mechanism 14, and a search engine 16. Data mart 12 is a data storage element which holds historical experimental data supplied from historical experimental database 18, chemical descriptor data from chemical descriptor database 20, and concurrent result data supplied from concurrent result database 22. Information from data mart 12 is provided to both point evaluation mechanism 14 and search engine 16. Search engine 16 supplies data to point evaluation mechanism 14, which in turn generates data for concurrent experimental result data storage 22. It is to be appreciated each of the components of hybrid learning system 10 may be implemented via a computing device where information within the system is maintained in a computer-readable format.

Point evaluation mechanism 14 includes supervised learning modules 24, 26, 28 and a scoring/filtering module 30. In this embodiment supervised learning modules 24, 26 and 28 may be one of many known neural networks or neural network equivalent techniques known in the art including but not limited to the various types of Classification/Decision Tree Analysis, Regression Analysis, and Principal Components Analysis. Regression Analysis includes not only classic types but also newer types such as General Additive Models and Multivariate Adaptive Regression Splines. Decision Tree Analysis includes not only traditional techniques such as CART and CHAID but also techniques such as networks of trees (Multivariate Adaptive Regression Trees) and Decision Tree Analysis with multiple responses.

Search engine 16 includes a genetic algorithm processor 32 and a clustering processor 34 such as a fuzzy clustering processor, which function in parallel. Other types of non-hierarchical or hierarchical clustering may be substituted for the fuzzy clustering processor, as may related techniques for classification and grouping such as Discriminant Analysis and Logistic Regression. Search engine output selector 35, may be provided to select at least one output from either processor 32 or 34, to be passed to point evaluation mechanism 30. Data from search engine 16 and unsupervised learning modules 24, 26, 28 supply data to scoring/filtering module 30. Information from scoring/filtering module 30 is used in determining which physical experiments 36 are to be performed. Data results from physical experiments 36 are supplied to concurrent experiment results database 22. The input to hybrid learning system 10 are experiments, while the output is a set of chemical elements that yield a highest turn over number (TON) and selectivity.

Through this construction, the hybrid learning system 10 enables an efficient search of an experimental space, such as a CC-space, using classification techniques and processes such as neural networks, genetic algorithms and clustering, among others.

Turning more particularly to data mart 12, this component is configured for the acquisition and easy manipulation of data regarding historical experiments, chemical descriptors, and concurrent experiments or other data which is relevant to a particular experimental space being investigated. Data mart 12 may include any one of known data access and storage techniques such as a relational database, which has standard query language capabilities or other manners of receiving requests or queries and responding thereto.

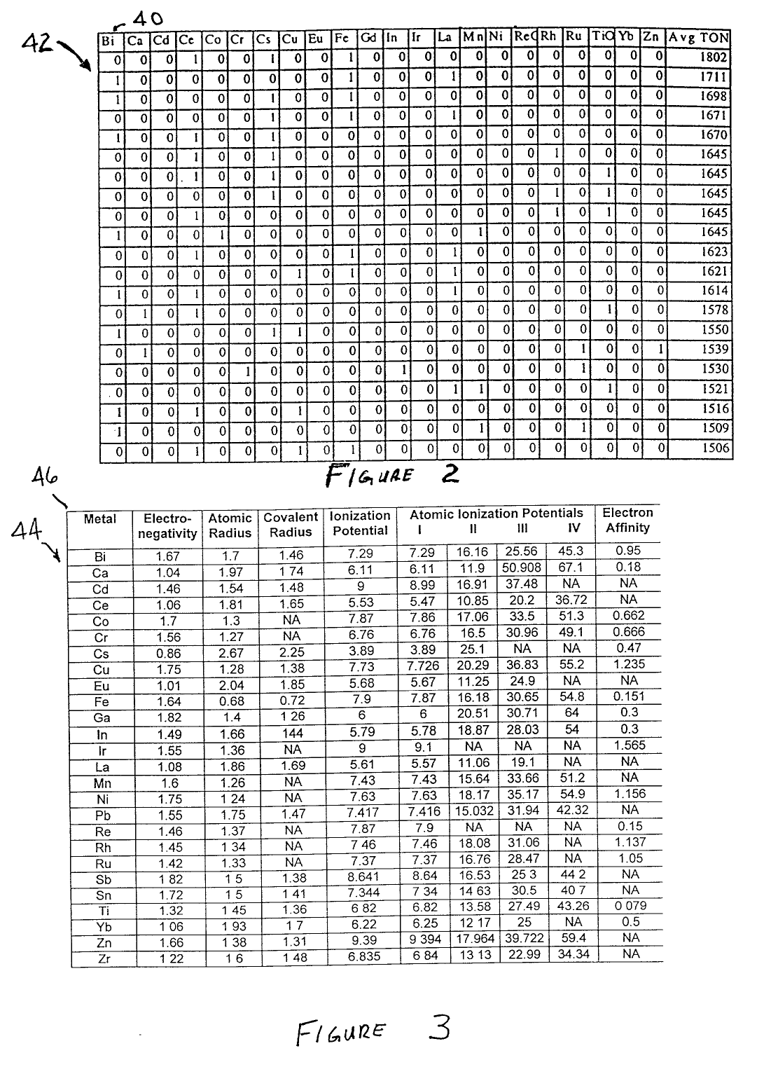

The main data sources, in this embodiment, include the experimental setups, historical experiment results, property descriptors of chemical elements, and current experiment results. Examples of such sample data for a triple catalyst run and chemical descriptors are illustrated in FIGS. 2 and 3 respectively. In FIG. 2 a row of chemical elements are listed 40. Below each element is a column 42 indicating whether or not that element is a catalyst of an experimental combination. A “1” represents that a catalyst has been added and a “0” indicates the catalyst has not been added for the experiment. FIG. 3 is a table having a column 44 of elements, where each element has a row of its chemical descriptors 46. This information may be stored in chemical descriptors database 20 of FIG. 1. As previously mentioned, such chemical descriptor data and data from a historical experiment database 18, and concurrent experiment results database 22 are inputs to data mart 12.

As a precursor to the creation of data mart 12, steps are undertaken to insure the integrity of each of databases 18-22. The creation of data mart 12 includes querying the various databases 18-22, to input data required for specific operation of hybrid learning system 10. As part of data mart creation, data scrubbing may be performed on results of queries made to databases 18-22. Such operations include detecting outliers, filling in or deleting missing values, and other techniques known in the art to generate a reliable source of information. It is to be understood that data mart 12 is a constantly evolving component which will for example include new experimental results from database 22 as they are produced.

Point evaluation mechanism 14 is configured to at least undertake physical experiments to yield a TON and selectivity, or to use a synthetic model to perform a supervised learning method (i.e. neural networks) to predict TON and selectivity, given a set of exploratory variables.

Search engine 16 uses both unsupervised learning techniques (e.g. clustering) and global techniques (e.g. genetic algorithms) to select a next set of experiments to undertake (i.e. the next set of points to evaluate). The function of search engine 16 is to find a next set of search points given a current position and a past search history.

Genetic algorithm processor 32 and clustering processor 34 operate in parallel, without needing to interact with each other. Therefore, search engine output selector 35 is designed to select at least one output from either processor 32, 34 to be passed on to point evaluation mechanism 30. The selected output may be based on a “best” output, where best is determined by a set of rules designed for a specific implementation. As an alternative embodiment, evaluation points selected by both processors 32, 34 may be passed to point evaluation mechanism 14 for further processing.

The concept of using clustering such as fuzzy clustering is to find the next set of search points that are most similar, but yet different from a current position. The advantage of fuzzy clustering is its fast convergence and easy to interpret results. However, fuzzy clustering is known to suffer from the fact that its solutions are in a sense homogeneous. For this reason, genetic algorithms are employed as a complement to the fuzzy clustering operations. The concept of using genetic algorithms is to take advantage of genetic algorithms ability to combine individual solutions to form even better solutions and its ability to escape from minimum points via mutation operators.

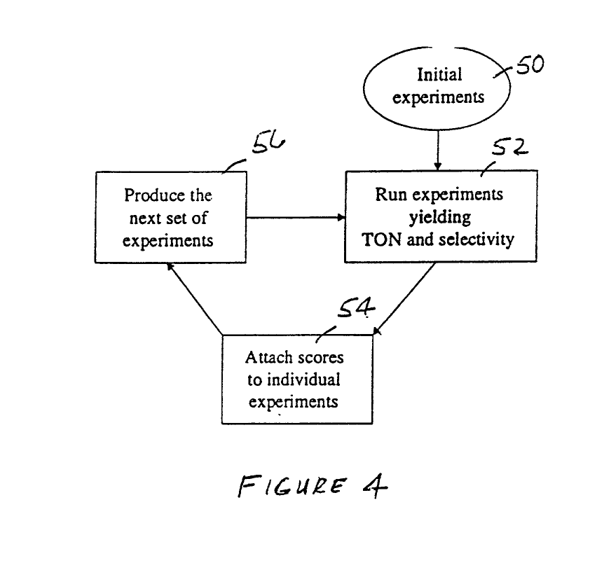

In searching a large experimental space, such as a CC-space, the combination of search management, which includes choosing a next set of experiments to undertake, the evaluation of points within the search space, and performing either the physical or synthetic experiments forms a cyclical flow of information such as represented in FIG. 4. This figure illustrates that initial experiments are selected 50, and these experiments are undertaken to obtain results, such as to yield TON and selectivity 52. The results of the individual experiments then have scores attached 54, and the scores are used in a decision-making process to produce the next set of experiments 56. The next set of experiments 56 are undertaken to again obtain experimental results 52. Thereafter, the individual experiments of this next cycle have scores attached 54. The process flow continues in the bottom loop between 52, 54 and 56, for a predetermined number of cycles until a reliable outcome is obtained. This outcome may be the solution to the problem, a potential solution, or may indicate the solution to the problem is not found within the test set.

As previously mentioned, the potential combinations to be investigated, i.e. experiments which may be undertaken, grows at an exponential rate resulting in an enormous experimental space (CC-space) which does not allow for an investigation of each point in the space.

Illustrating the above concept, FIG. 5 depicts a CC-space 60. Each point 62 within space 60 correlates to a potential experiment which may be undertaken to determine an output. It is to be appreciated that CC-space 60 of FIG. 5 is only a fraction of a full CC-space. Further, CC-spaces have a very high dimensionality where not just one, two or three dimensions exist, but rather ten, twelve, seventeen or more dimensions may exist within a space. Also, there is no real required relationship between the dimensions so in a sense points within a CC-space are each a set of discrete points. This creates further difficulties in investigating such a space. Thus, a goal of the present invention is to find an efficient effective manner to investigate small fractions of a CC-space which nonetheless will provide highly reliable outputs as to the solution of an investigation or determination that the solution does not exist within the CC-space.

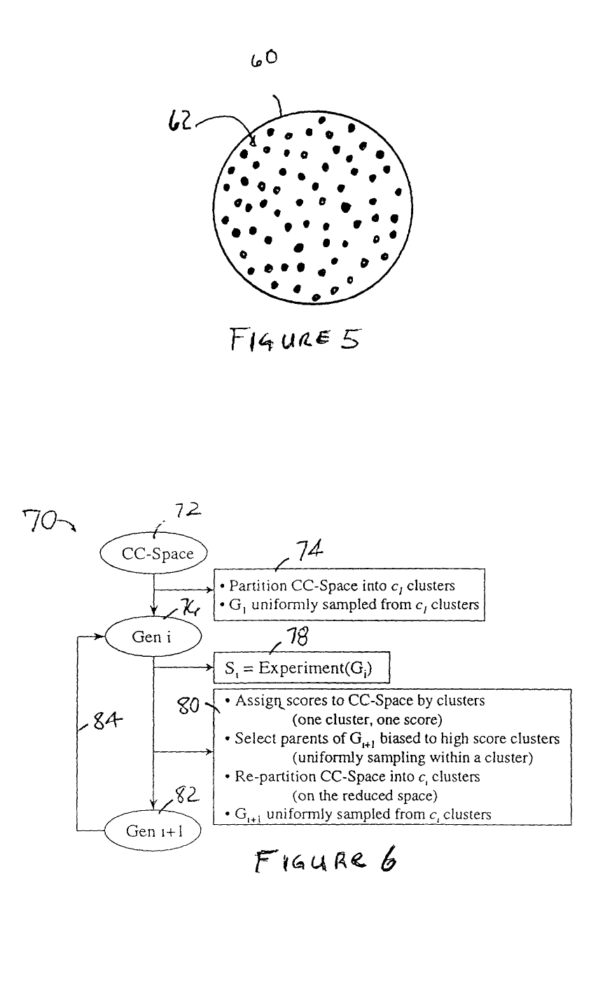

FIG. 6 depicts a flow diagram 70 of an unsupervised learning process for CC-space exploration. Once the CC-space is defined 72, it is partitioned into clusters of points having similarities 74. Clustering does not initially address itself to finding a solution of an experiment, but rather arranges the CC-space into a design where like points are provided within a particular cluster. Therefore, points within a particular cluster (or sub-space) of the CC-space are highly correlated to each other. Likeness may be defined on a per application basis. One example may be points are clustered in accordance with the largest individual element of a combination of elements.

Instead of performing an experiment on all points, the CC-space is uniformly sampled on a cluster basis to obtain representative points to be tested. The sampling may be a random process, within a cluster. This collection of representative points are the first generation (GEN i) 76 of points which are to have experiments performed on them (Sᵢ=experiment (Gᵢ)) 78. After running of the experiments (physical experiments or synthetic experiments), each cluster will be given a score as determined by the experiment on the selected point or points from the cluster. Thereafter parents are selected upon the basis of a score of a cluster. The CC-space is repartitioned into clusters on a reduced space. Next, a selection is made of a second generation of points and there is a uniform sampling from the remaining clusters 80. The system is further designed to move from the present generation of points 82, and loop back 84 to continue the process.

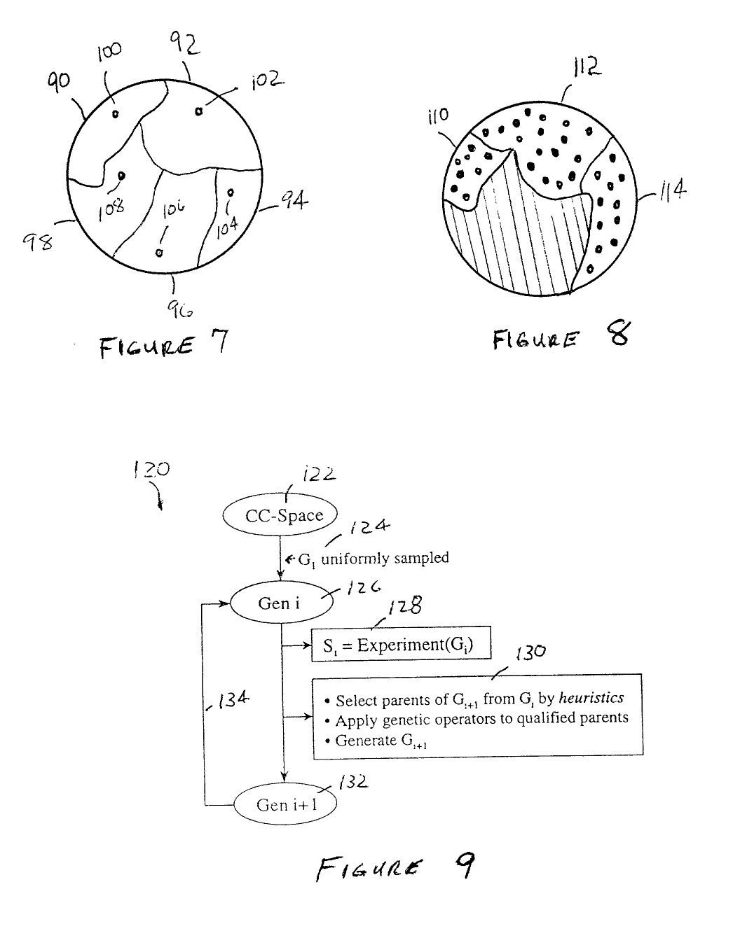

The operation of the cluster processing by the clustering processor 34 on CC-space 60 of FIG. 5 is illustrated more particularly in FIGS. 7 and 8. The following discussion also correlates to the flow diagram of FIG. 6. As an initial step, in reviewing the CC-space 60, the clustering processor 34 uses existing historical experimental data as well as information regarding the chemical elements and their properties and functions under a paradigm that points located near determined “good” points should themselves be good. Based on this philosophy, the clustering process divides the CC-space 60 into a number of clusters 90-98. Thereafter, from within these clusters the clustering process selects a small number of points (e.g. one or two) to generate a first generation (Gen i of FIG. 6) 100-108 on which experiments are to be performed. By this arrangement, the clustering processor greatly reduces the number of experiments within a CC-space, with one or two points within one of clusters 90-98 representing that space. This is called an unsupervised learning algorithm since the first step of the algorithm does not care about the results of the experiment, rather clustering is done in accordance with similar points within CC-space 60. Once points within the clusters are obtained, the experiments or synthetic modeling of experiments may be undertaken as to the selected points (e.g. Sᵢ=Experiment (Gᵢ); of FIG. 6). Based on these experiments or experiment modeling, scores are assigned to the clusters 90-98 (i.e. one cluster will obtain one score). Thereafter parents (Gᵢ₊₁) are selected based on the score obtained by a cluster.

Using this information, clusters with certain scores will be determined to be undesirable (e.g. clusters 96 and 98 of this example). Thereafter, a repartitioning of the CC-space into clusters (Cᵢ of FIG. 6) 110, 112, 114 will be undertaken, and a uniform sampling within clusters 110, 112, 114 is performed to obtain a next generation of points to be evaluated (Genᵢ₊₁). At this point, system 10 can cycle back through the process of experimentation and repartitioning of the CC-space to further refine the search space. Alternatively, the data may be supplied to the point evaluation mechanism 14.

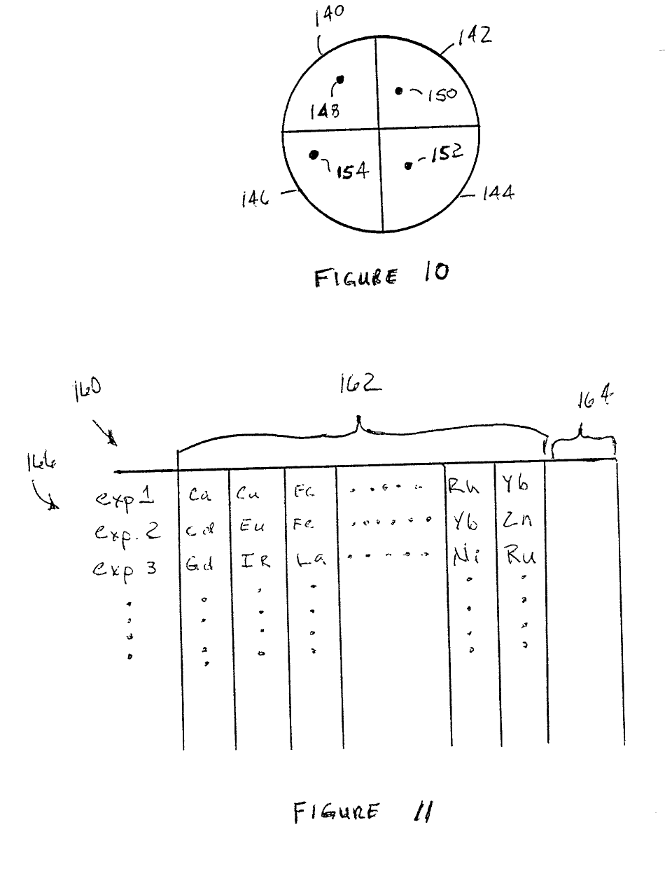

FIG. 9 illustrates a flow diagram 120 for a genetic algorithm process for CC-space exploration. Initially the CC-space 122 is uniformly sampled 124, where one or two points from each section from the CC-space is selected. This creates a pool of potential points (Gen i) 126. These points are then evaluated (Sᵢ=experiment (Gᵢ)) 128. This experiment may be an actual physical experiment or undertaken using an experimental model. Therefore, the individual points represent whether or not the subspace from which it was drawn is good or bad. Through this process, good subspaces may be selected. The next step includes selecting the parents of the generation from Gᵢ. The majority of parents selected are classified as “good” parents, i.e. they are good or acceptable points. However, the parents may also chosen probabilistically to allow the possibility of a bad parent to be chosen. The reason for this is in order to maintain diversity. However, probability of selecting a good parent is much greater than the selection of a bad parent. Selection of parents theoretically works toward producing a more acceptable offspring.

The selection of the parent may be done by heuristics, where in a first step selects what are to be considered “good” parents. The selection of a good parent is based on a set of predetermined rules, and the selection creates the next generation of potential test points 130. Using the obtained generation of points (Genᵢ₊₁) 132, the selection process can be repeated 134 to obtain a desired grouping of points having a higher values returned by the fitness function.

An issue with genetic algorithms, however, is that if only good parents (good points) are used, some diversity in the selection process may be lost. Then, no matter how the parents are combined, large portions of a subspace will be excluded from exploration. Therefore it is desirable to have some diversity which is the exploration part of an exploration/exploitation issue in any genetic algorithm, where exploitation is directed to obtaining the best possible choice as quickly as possible.

It is noted that when the genetic algorithm is functioning, there is an intermediate stage of the solution. It is possible to produce a larger population of potential parents than the existing population. The question becomes how is the larger population evaluated to obtain only a desired number (e.g. 110) experimental points. In this embodiment, 110 points are selected as it is presently the largest number of physical experiments which can be undertaken at one time.

So the number of points which can be handed off to the physical experimental stage is a maximum of 110 being done at one time. It is possible, however, to produce a larger in-term population and that population can then be whittled down to the 110 experiments.

Initial data is supplied to the genetic algorithm processor 32 in a manner similar to that supplied to the clustering processor 34. As may be seen by FIG. 10, the genetic algorithm processor 32, however, uniformly partitions CC-space 60 into substantially equal spaces or sections 140-146. Thereafter, one or two sample points from each section 148-154 are selected as the initial generation of points (Gen i). Thereafter, experiments (Sᵢ=Experiment (Gᵢ)) are performed on the select data points. Parents are selected from the output by heuristics. Genetic operators are applied to qualified parents to generate a next generation (GENᵢ₊₁). This process may be repeated to refine the potential pool of points to be investigated. Alternatively, the process may be provided to the point evaluation mechanism 14 as shown in FIG. 1.

Point evaluation mechanism 14, uses supervised learning techniques such as neural networks to implement models of a fitness function making it possible to evaluate, for each one of the potential children, what an expected score would be. These scores are then to be used to describe which of the points are to be used for physical experiments.

As previously noted, the time period to run a single cycle through a physical experimental loop, such as shown in FIGS. 1 and 4, is dominated by the time required to execute the complete experimental phase of the cycle. Such experiments commonly may take up to a week. Thus, this is a bottleneck of CC-space searching. The concept of building point evaluation mechanism 14 is to approximate the chemical reaction involved in a particular experiment. A function as used here is developed to approximate a fitness function being computed in the chemistry.

This fitness function may be defined as:

y=f(x),

and is more particularly concerned with finding what function of x provides a desired or useful y.

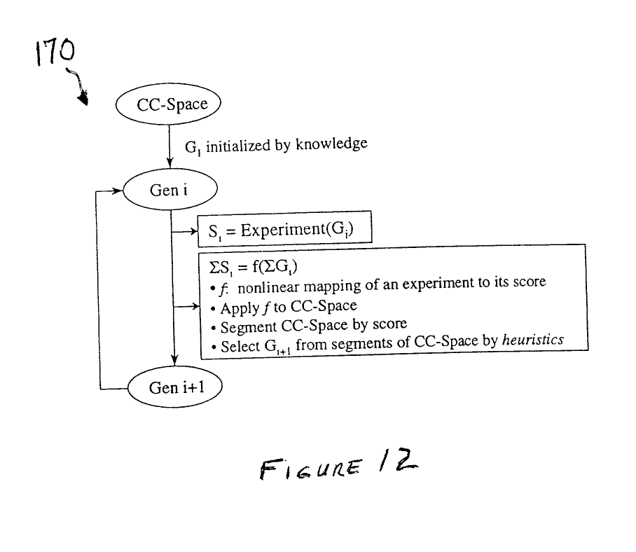

Within the Combinatorial Chemistry field, x may be a representation of the various chemicals and properties being tested, and y the average TON of a particular x. For example, turning to FIG. 11, in table 160, column 162 lists the chemical elements and properties of the chemical elements within the CC-space (i.e. x). Column 164 represents an output (i.e. y). These chemicals and properties may be taken from tables such as those of FIGS. 2 and 3. Each line 166 in table 160 represents an experiment which may be performed to determine what the function (f) produces as an output (y).

In place of actual physical experiments, point evaluation mechanism 14 implements supervised learning techniques, such as neural networks, in modules 24, 26 and 28 to obtain a continually improving approximation for the fitness scores for experiments which are performed.

For example, it is assumed that an estimate of the fitness function after t cycles of a search loop (genetic or clustering) has been obtained. This estimate will be called f′. Next, some subset e of potential candidates will be selected. These candidates may be randomly chosen. Next, the best x in subset e will be chosen, where the “goodness” of x is given by f′(x). The experiments will then be performed, yielding f(x) for each of these points. A new estimate f′⁺¹ is then derived from f′, x, and f(x). The derivation of a new estimate for f is where a variety of supervised learning techniques are applied.

Returning attention to FIG. 3, the elements or properties of the chemicals may be found by searching the literature or by doing quantum mechanical calculations, well known in the art, or by doing experiments to determine properties which are considered as possibly being related to the y being investigated. The concept being that if its possible to relate the x's which are the properties of these values, there exists a better chance of finding f′ (which is a model of f).

When it is mentioned that selected points represent experiments which may be undertaken to solve for y, it is intended to be understood number of potential solutions available to obtain a desired or good output.

Turning to FIG. 12, depicted is a flow diagram 170 of a supervised learning process for CC-space. By using the data from tables such as tables shown in FIGS. 2 and 3, historical data known for this experiment can be collected and used by the supervised learning modules 24, 26, 28 to formulate a model function (f′) which attempts to approach function (f).

If this process is thought of as a linear equation, plotted, it will be x against y with many points between x and y. The data point for f represents one straight line. Ignoring everything else, that straight line would represent the f function. Therefore finding the straight line for f, is what is being attempted by the supervised learning modules, which is to determine a model of the f function. Therefore, using supervised learning modules 24, 26, 28, if the initialized knowledge (i.e. the prior known knowledge) is used, a best estimate as to what is the f function, without requiring a physical experiment is attempted to be found.

System 10 moves from a first generation of points to a next generation using standard genetic algorithm or clustering approaches. From this operation a somewhat larger population than the initial population may be obtained. It is therefore necessary to decide what is to be done with that population. Data from previously undertaken experiments can be used with the supervised learning modules, to build a model against which the proposed experiments can be run. These models can, in fact, be kept in parallel in each of modules 24, 26 28. More weight can be provided to the model (one of 24, 26, 28) which gives the best results. However, in the beginning of the operation, it is not known which model might be most effective, therefore they are all weighted the same. However, as actual data is returned, a comparison to the model data of each module 24, 26, 28 will determine which model is more efficient or accurate. The system 10 then gives that model more weight to its output. The model with the highest weight produces the best approximation (f′) to y.

Weighting of the models for each module 24, 26, 28 is accomplished via scoring mechanism 30. The scoring mechanism 30 may include any of a number of criteria (e.g. one including the highest scored points being passed on to determine the weighting). Nevertheless, the supervised modules 24, 26, 28 are used to generate f′ functions which are then scored from best to worst. From the operation points are selected for physical experiments. Using the physical experiment data, the models of the supervised modules 24, 26, 28 are updated using the new data added to the database of the data mart 12.

With the new population of points which have been developed, the system again goes through the genetic loop performed by the genetic algorithm processor 32 and in parallel the clustering loop, performed by the clustering algorithm 34. The obtained points, from either the genetic algorithm processor 32 and the clustering processor 34, or both, are then supplied to all or some of modules 24, 26, 28 in order to compare the newly acquired points with the newly refined models.

Therefore, the hybrid nature of the present invention is two-fold. First, is the idea of using the genetic algorithm and/or clustering to be tied to a physical experiment. The second is the building of better and better approximations as more data is gathered to predict what the experiment is going to do. This makes it possible to virtually explore a larger space each time than the number of experiments that are going to be performed. When the process cycles through for a second time, while the CC-space itself will stay constant, the area being investigated within the CC-space may increase in size or decrease dependent upon the absolute use of the scores. If only the best scores are used, then the CC-space being investigated is narrowed. If outliers or some of the “non-best” parents are used such as by probability processes, then the space does not necessarily decrease at a quick pace. It is desirable to control the pace of focusing in on a best answer such that areas of the CC-space are not overlooked.

Upon an initial operation of the system 10 points being investigated are well distributed over the CC-space. However, over time the points being investigated will tend to concentrate within a particular area.

For example, assuming a space of 100%, where 10% of the space is going to be “good space” and 90% of the space will be “bad space”, upon an initial operation of system 10, approximately 10% of the points investigated will be in the “good space” and 90% in the “bad space.” However, after a number of operations of the system 10, the number of points in the good space would be increasing and the number of points in the bad space decreasing. For example, after 20 cycles of the system, 50% of the points will be in the good space and 50% in the bad space. Then after 40 or 50 generations, maybe 90% of the points will be in the good space and 10% of the points will be in the bad space. This maintaining of the bad points allows for the system to consolidate in an efficient manner while insuring that areas are not being overlooked. At some point, for example, when 90% of the points are in the good space, it may be determined that enough testing has been done and the final outcome is derived.

In common operation, 1 or 2 random loops of genetic algorithm processor 32 and/or clustering processor 34 are undertaken to obtain a base population of points. Then the genetic algorithm loop 32 and/or clustering loop 34 performing, along with the model approximation learning loop (24, 26, 28) meet at the scoring/filtering section 30 to determine which experiments are to be performed. The scoring mechanism can be based on any one of many factors, including what is a commercially valuable result which is to be obtained. For example, it can be a catalyst yield or activity, a coating barrier quality or other commercially valuable results. Therefore the basic scoring mechanism which is to be learned will be defined by the application.

Using the present embodiment of the invention, a rational logical system is disclosed to obtain a conclusion of complex problems, either leading to a solution, potential solutions which may be further investigated, or to the conclusion the CC-space being investigated does not contain the potential solutions.

While the invention has been described in conjunction with the specific embodiments thereof, it is evident that many alternatives, modifications and variations will be apparent to those skilled in the art in light of the foregoing description. Accordingly, the present invention is intended to embrace all alternatives, modifications and variations which fall within the spirit and broad scope of the appended claims.

1. A hybrid learning system for searching an experimental space, comprising:
a data mart configured to acquire, store and manipulate at least, historical experimental data, descriptor data, and concurrent experimental data;
a search engine configured to use selection techniques to select a set of evaluation points representing a corresponding set of experiments to be run, based on the data from the data mart; and
a point evaluation mechanism configured with
(i) learning modules which perform predictive processing on the evaluation points selected by the search engine, and
(ii) a scoring module which performs a rating operation on outputs of the learning modules to rate the outputs of the learning modules,
wherein operation of the data mart, search engine and point evaluation mechanism are operated a plurality of times such that a repeating process is undertaken to obtain a finalized output.

2. The system according to claim 1 further including a physical experiment, wherein results of the physical experiment are supplied to the data mart.

3. The system according to claim 2 wherein the experimental space is a Combinatorial Chemistry experimental space.

4. The system according to claim 3 wherein an input to the system are experiments and the output of the system is a set of elements that yield a highest turnover number (TON) and selectivity.

5. A method for exploring an experimental space using a hybrid learning system, the method comprising:
(a) generating an experimental space including a plurality of experimental points, representing potential solutions to an experiment;
(b) collecting historical experimental data, descriptor data, and concurrent experimental data;
(c) storing the historical experimental data, descriptor data, and concurrent experimental data in a data mart, wherein the data mart includes the ability to be queried;
(d) performing a genetic algorithm processing loop on the experimental space to obtain a subset of experimental points from the plurality of experimental points;
(e) performing a clustering processing loop on the experimental space to obtain a subset of experimental points from the plurality of experimental points;
(f) selecting the subset of experimental points from at least one of the genetic algorithm processing step and the clustering processing step;
(g) supplying the selected experimental points and a subset of the data from the data mart to a point evaluation mechanism;
(h) performing a supervised learning process on the selected points; and
(i) obtaining an output.

6. The method of claim 5 further including performing a physical experiment using experimental points from the experimental space to obtain actual physical experimental results.

7. The method of claim 6 wherein the physical experimental results are supplied to the data mart.

8. The method according to claim 5 wherein steps (b)-(i) are repeated.

9. The method according to claim 5 wherein the experimental space is a Combinatorial Chemistry experimental space.

10. The method of claim 5 wherein the clustering loop includes:
(a) partitioning the experimental space into clusters of points having similarities;
(b) selecting a sample from each cluster, the sample being at least one evaluation point, wherein the selected samples are a first generation of evaluation points;
(c) performing at least one of actual physical experiments or synthetic models of experiments using the first generation of evaluation points;
(d) scoring each cluster based on an outcome of the at least actual experiment and synthetic models;
(e) selecting a cluster based on the scoring;
(f) repartitioning the experimental space into clusters on a reduced space; and
(g) repeating steps (b)-(f).

11. The method of claim 5 wherein genetic algorithm loop includes:
(a) partitioning the experimental space into uniform spaces of points;
(b) selecting a sample from each uniform space, the sample being at least one evaluation point, wherein the selected samples are a first generation of evaluation points;
(c) performing at least one of actual physical experiments or synthetic models of experiments using the first generation of evaluation points;
(d) scoring each uniform space based on an outcome of the at least actual experiment and synthetic models;
(e) selecting points to be parents based on the scoring;
(f) generating a next generation of points based on selected parents; and
(g) repeating steps (b)-(f).

12. The method according to claim 5 wherein each time a set of experiments is performed, additional data is added to the system and a further refined model is generated.

13. The method according to claim 5 wherein the selection processes are run against a new improved model.

14. A hybrid learning system for searching an experimental space comprising:
a data mart configured to receive, store and supply data;
a search engine including at least a genetic algorithm processor and a clustering processor configured to operate in parallel, both the genetic algorithm processor and the clustering processor configured to request data from the data mart, in order to select a set of points from the experimental space, the points representing a corresponding set of experiments to be undertaken; and
a point evaluation mechanism including at least one learning module and a scoring module, the at least one learning module receiving data from the data mart and the search engine and having a model experiment to which the selected points and data are applied.
